(19) United States
(12) Patent Application Publication
(10) Pub. No.: US 2004/0030127 A1
(43) Pub. Date: Feb. 12, 2004

(54) Process for the preparation of highly crystalline sodium cefoperazone

(76) Inventors: Walter Cabri, Rozzano (IT); Giovanni Pozzi, Besana Brianza (IT); Paolo Ghetti, Segrate (IT); Domenico Vergani, Villasanta (IT); Roberto Strigaro, Milano (IT)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/240,758

(22) PCT Filed: Apr. 10, 2001
(86) PCT No.: PCT/EP01/04074

(30) Foreign Application Priority Data
Apr. 14, 2000 (IT) MI2000A000837

(51) Int. Cl. C07D 501/14

(52) U.S. Cl. 540/222

(57) ABSTRACT

Highly crystalline, highly filterable sodium Cefoperazone in the form of needle crystal aggregates, obtainable by a process comprising the controlled addition of acetone to a solution of water/acetone/alcohols/sodium Cefoperazone at 20˜40° C.

The present invention relates to a process for the preparation of sterile, highly crystalline sodium Cefoperazone 7-[[[[(4-ethyl-2,3-dioxo-1-piperazinyl)carbonyl]amino](4-hydroxyphenyl)-acetyl]-amino]-3-[[(1-methyl-1H-tetrazol-5-yl)thio]methyl]-8-oxo5-thia-1-azabicyclo[4.2.0]oct-2-ene-2-carboxylic acid, monosodium salt, [6R-[6α,7β-(R*)]]), in the form of needle microcrystal agglomerates which are a further object of the invention.

Sodium Cefoperazone, disclosed in U.S. Pat. No. 4,087,424 (May 1978), is an injectable semi-synthetic cephalosporin with broad spectrum activity against resistant Gram negative bacteria, widely used in clinic due to its effectiveness in the cases of severe infections of skin, high and lower respiratory tract, central nervous system and genital-urinary system.

Sterile sodium Cefoperazone is available both in the amorphous (GB 1,508,071) and crystalline form (U.S. Pat. No. 4,458,070, Jul. 1982). The crystalline product, obtained by crystallization, has higher purity, higher chemical stability, and lower hygroscopicity than the amorphous product, usually obtained by freeze-drying. The preparation of the product in the crystalline form is disclosed in U.S. Pat. No. 4,458,070 and in U.S. Pat. No. 4,456,753: U.S. Pat. No. 4,458,070, (Jul. 1982) comprises the crystallization of sodium Cefoperazone from a water/acetone/methylene chloride mixture, whereas U.S. Pat. No. 4,456,753 (Feb. 1983) discloses an improved process providing a “highly crystalline” product from the acetone/water mixture, thus avoiding the use of a toxic solvent such as methylene chloride (class 1, ICH guidelines). This method, however, provides highly crystalline sodium Cefoperazone in the form of free needle microcrystals. It is therefore difficult to filter or centrifugate the resulting suspension with the apparatuses usually employed for the preparation of sterile bulk, due to the compressibility of the product and its tendency to pack. As a consequence, drying is carried out with detriment of the process productivity and costs.

### SUMMARY OF THE INVENTION

We have now surprisingly found a process for the preparation of Cefoperazone in the form of needle microcrystal agglomerates, which are easy to filter and to dry, by crystallization of the sodium salt from water/acetone mixtures in the presence of alcohols and/or mixtures thereof. The process of the present invention is therefore characterized by high productivity on the industrial scale. The resulting sodium Cefoperazone is still as highly crystalline as that disclosed in U.S. Pat. No. 4,456,753.

### DETAILED DISCLOSURE OF THE INVENTION

The process of the present invention comprises the salification of acid Cefoperazone in water/acetone mixtures with one equivalent of a base selected from sodium bicarbonate, sodium carbonate and sodium 2-ethylhexanoate, keeping the temperature at 5 to 25° C. The sodium Cefoperazone solution is added with C₁-C₄ aliphatic alcohols or mixtures thereof at temperatures of 20° C. to 40° C. Cefoperazone sodium is crystallized by addition of acetone, keeping the temperature at 20 to 40° C.; said addition is preferably carried out in two portions: the first portion is sufficient to form a sodium Cefoperazone suspension, the second portion allows to complete the crystallization to a low content in sodium Cefoperazone in the mother liquors. The filtration or centrifugation of the product can be carried out after 90 minutes or one day after the end of the crystallization, at the crystallization temperature (20 to 40° C.) or after cooling the suspension to temperatures of 0 to 20° C. The crystalline product is dried under vacuum (20 to 300 mbars) adjusting the temperature of the product to 35-40° C.

Acid Cefoperazone is salified by using preferably water/acetone/Cefoperazone ratios of 0.8-0.6/1,4-5,0/1,0 v/v/w with one equivalent of a base, preferably sodium bicarbonate, in amounts of 0.124˜0.130 grams/1 gram of acid Cefoperazone, thereby obtaining water/acetone/sodium Cefoperazone solutions containing 19˜35% of water at a temperature of 18˜22° C. The sodium Cefoperazone solution is added with alcohols or mixtures thereof selected from methanol, ethanol, n-propanol, n-butanol and isopropanol at temperatures of 20° C. to 40° C., preferably at 23 to 30° C. Ethanol (0.2-0.4 ml per 1 gram of acid Cefoperazone) or isopropanol (0.2-0.8 ml per 1 gram of acid Cefoperazone) or mixtures thereof (ethanol/isopropanol: 0.2-0.4/0.2-0.8 ml per 1 gram of acid Cefoperazone) are preferably used. Sodium Cefoperazone is crystallized by adding acetone in two portions, keeping the temperature at 20 to 40° C., preferably at 23 to 30° C. The first acetone portion, sufficient to form a Cefoperazone sodium suspension, provides a water/acetone mixture containing 11 to 14% of water. The second portion provides complete crystallization which is attained when the water/acetone mixture has water content of 3 to 5%. The product may be filtered or centrifuged either 90 minutes after the end of the crystallization, at a crystallization temperature of 20 to 40° C., or after cooling the suspension to temperatures of 0 to 20° C. The wet product is first washed with water/acetone mixtures containing 2 to 5% of water, and finally with pure acetone. The crystalline product is dried under vacuum at 20 to 300 mbars and at 35˜40° C.

The process of the invention yields crystalline sodium Cefoperazone in the form of needle microcrystals where at least 50% of the aggregates have diameter above 10 μm and at least 10% of the aggregates have diameter above 150 μm (particle size distribution was determined by laser diffraction on the dry product).

### DESCRIPTION OF THE FIGURE

FIG. 1 is an optical microscope photograph of the crystalline sodium Cefoperazone in the form of needle free microcrystals obtained from acetone without using alcohols, according to U.S. Pat. No. 4,456,753 (example 2).

FIGS. 2, 3 and 4 are optical microscope photographs of the crystalline sodium Cefoperazone in the form of needle microcrystal agglomerates obtained from acetone in the presence of alcohols according to the following examples 1-3, respectively.

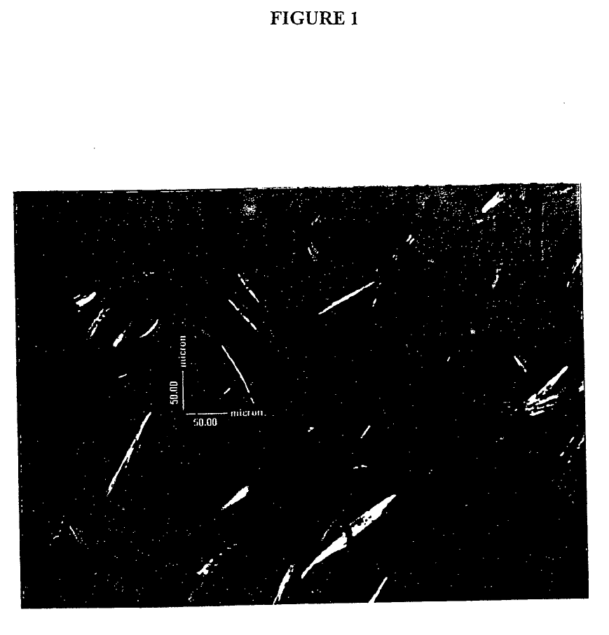

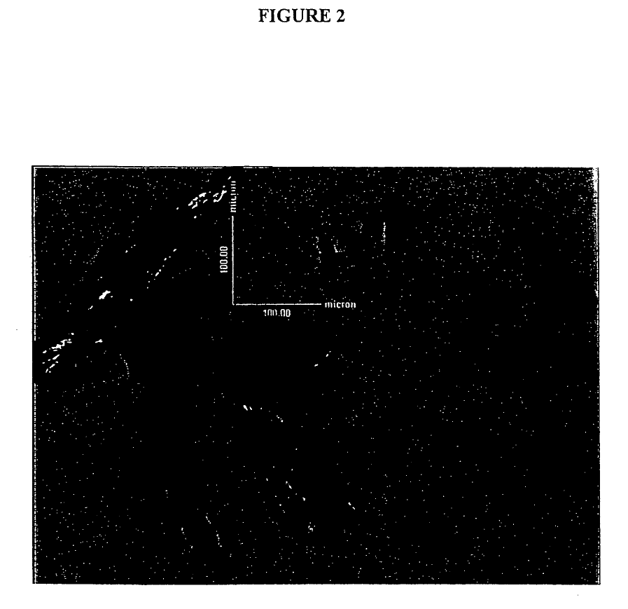

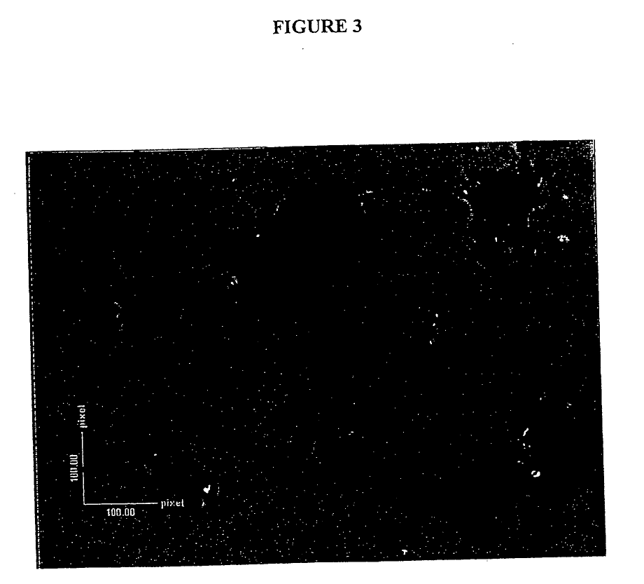

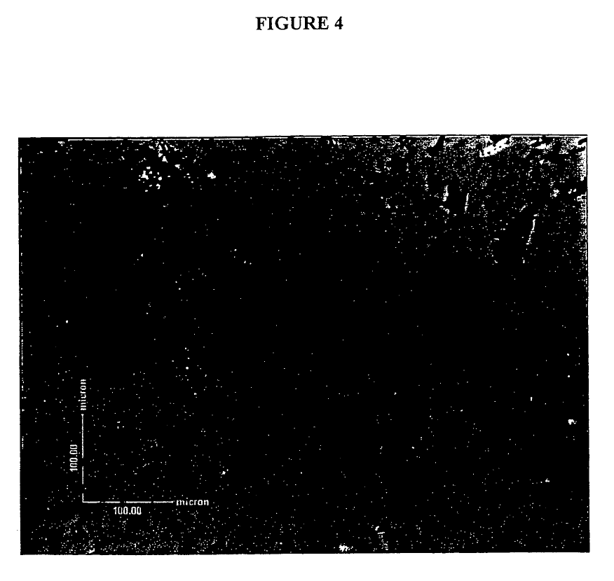

### EXAMPLES

The following examples illustrate the invention in greater detail.

### Example 1

### Crystallization of Sodium Cefoperazone in the Presence of Ethanol

To a suspension of 39.45 grams of acid Cefoperazone in 118.8 ml of acetone at 20-25° C., under vigorous stirring, a solution of 4.79 grams of sodium bicarbonate in 59.4 ml of purified water was added. The sodium Cefoperazone solution, pH 5.9-6.0 at 25° C., was diluted with 8 ml of absolute ethanol without formation of precipitate. Acetone, 269 ml, was added under stirring at 25° C. in 30 minutes until slight, but persistent turbidity. The suspension was stirred for 5 minutes at 25° C. until intense turbidity, then 1013 ml of acetone were added dropwise in 20 minutes. After stirring for 60 minutes at 25° C., the product was recovered by filtration under vacuum. The crystal was washed first with a mixture of 15 ml of water and 323 ml of acetone, then with 280 ml of pure acetone. After drying for 15 hours under 1 mbar at 35° C., 36.90 grams of crystalline sodium Cefoperazone were obtained.

### Example 2

### Crystallization of Sodium Cefoperazone in the Presence of an Ethanol/Isopropanol Mixture

To a suspension of 39.45 grams of acid Cefoperazone in 118.8 ml of acetone at 20÷25° C., under vigorous stirring, a solution of 4.79 grams of sodium bicarbonate in 59.4 ml of purified water. The sodium Cefoperazone solution, pH 5.9÷6.0, at 25° C. was diluted first with 8 ml of absolute ethanol then with 16 ml of isopropanol without formation of precipitate. Acetone, 269 ml, was added under stirring at 25° C. in 25 minutes until slight, but persistent turbidity. The suspension was stirred for 5 minutes at 25° C. until intense turbidity, then 1013 ml of acetone were added dropwise in 20 minutes. After stirring for 60 minutes at 25° C. the product was recovered by filtration under vacuum. The crystal was washed first with a mixture of 15 ml of water and 323 ml of acetone, then with 280 ml of pure acetone. After drying for 15 hours under 1 mbar at 35° C., 37.65 grams of crystalline sodium Cefoperazone were obtained.

### Example 3

### Crystallization of Sodium Cefoperazone in the Presence of Isopropanol

To a suspension of 39.45 grams of acid Cefoperazone in 118.8 ml of acetone at 20÷25° C., under vigoruos stirring, a solution of 4.79 grams of sodium bicarbonate in 59.4 ml of purified water was added. The sodium Cefoperazone solution, pH 5.9÷6.0 at 25° C., was diluted with 16 ml of isopropanol without formation of precipitate. Acetone, 269 ml, was added under stirring at 25° C. in 25 minutes until slight, but persistent turbidity. The suspension was stirred for 60 minutes at 25° C. until intense turbidity, then 1013 ml of acetone were added dropwise in 20 minutes. After stirring for 60 minutes at 25° C. the product was recovered by filtration under vacuum. The crystal was washed first with a mixture of 15 ml of water and 323 ml of acetone, then with 280 ml of pure acetone. After drying for 15 hours under 1 mbar at 35° C., 37.52 grams of crystalline sodium Cefoperazone were obtained.

1. A process for the preparation of crystalline sodium Cefoperazone in the form of needle microcrystal aggregates, which comprises the crystallization of sodium Cefoperazone from a mixture of water, acetone and C1-C4 aliphatic alcohols.

2. A process as claimed in claim 1 wherein the aliphatic alcohols are selected from ethanol, isopropanol or mixtures of ethanol and isopropanol.

3. Crystalline sodium Cefoperazone in the form of needle microcrystal aggregates obtainable by the process of claims 1-2.

4. Crystalline sodium Cefoperazone as claimed in claim 3, where at least 50% of the aggregates have diameter above 10 μm and at least 10% of the aggregates have diameter above 150 μm.
